(19) United States
(12) Patent Application Publication
(10) Pub. No.: US 2004/0241659 A1
(43) Pub. Date: Dec. 2, 2004

(54) Apparatus and method for hybridization and SPR detection

(75) Inventors: Steven J. Cox, Richmond, CA (US); Janusz B. Wojtowicz, Palo Alto, CA (US); Douwe D. Haga, Redwood City, CA (US); Mark F. Oldham, Los Gatos, CA (US); Michael W. Recknor, Oakland, CA (US); Gary P. Schroth, San Ramon, CA (US); Tracy L. Ferea, Mountain View, CA (US)

Correspondence Address: MILA KASAN, PATENT DEPT., APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 10/448,803

(22) Filed: May 30, 2003

(51) Int. Cl. C12Q 1/68; C12M 1/34

(52) U.S. Cl. 435/6; 435/287.2

(57) ABSTRACT

An apparatus and method for hybridization providing a chamber for hybridization and SPR detection. The hybridization chamber can have an inlet port and an outlet port. The chamber is adapted for SPR detection by a grating or by prism.

### CROSS-REFERENCE TO RELATED APPLICATIONS

Cross-reference is made to co-pending U.S. Non-Provisional patent Application No. ______ entitled “Apparatus and Method for Hybridization” to Wojtowicz et al. (Attorney Docket No. 5091), filed concurrently herewith; which is incorporated herein by reference.

### FIELD

The present teachings can be related to apparatuses and methods for conducting reactions on a solid surface with biological samples, including hybridization assays. The apparatus improves the process of hybridization and detection.

### BACKGROUND

In the biological field, reactions on a solid surface can be used for hybridization assays. A known member of a binding pair on the solid surface can hybridize with a target member of the binding pair from the biological sample to form a duplex in the hybridization fluid. A pattern of duplexed binding pairs on the solid surface provides information about the biological sample. The pattern on the solid surface can be detected to map the information relative to the known members of the binding pairs on the solid surface. It is desirable to control the volume over the solid surface during hybridization and to provide access to the solid surface during detection.

It can be desirable to have a sealed hybridization chamber to reduce leaks or evaporation of the hybridization fluid during processing and to maintain precise control over reaction temperature, duration, mixing, and other hybridization parameters. Hybridization parameters can include elevated temperatures, such as 55° C., for 18-24 hours with 400 rpm agitation. It can be desirable that a housing sealing the hybridization chamber can provide the container for shipping the hybridization chamber that can be sealed from the environment.

### SUMMARY

According to various embodiments, an apparatus for hybridization can include a substrate and a hybridization chamber including a detection frame, a hybridization frame, and a portion of the substrate, wherein the detection frame is positioned on the substrate, and the hybridization frame is positioned proximate to the detection frame to form the hybridization chamber. According to various embodiments, an apparatus for hybridization can include a housing including a base and a cover, a substrate including a microarray, wherein the substrate is positioned within the base, and a hybridization chamber including a frame and a portion of the substrate, wherein the cover releasably mates with the base to seal the hybridization chamber, and wherein the cover includes an inlet port to prevent a filling device from contacting the microarray.

According to various embodiments, a method for hybridizing can include providing a housing including a base and a cover, providing a substrate including a microarray, providing a hybridization chamber including a frame and a portion of the substrate, and releasably mating the cover to the base to seal the hybridization chamber and compress the frame.

According to various embodiments, an apparatus for hybridization can include a housing including a base and a cover, wherein the base and the cover are releasably mated, a substrate including a microarray, wherein the housing is configured to house the substrate, a detection frame, wherein the detection frame is positioned on the substrate, and the detection frame and the substrate form a detection volume, and a hybridization frame, wherein the hybridization frame, the substrate and the cover form a hybridization volume, wherein the hybridization volume and the detection volume are in fluid contact with the microarray. According to various embodiments, an apparatus for hybridization can include a substrate including a microarray, wherein the substrate includes a binding layer and a support, and a hybridization chamber including a frame and a portion of the substrate, wherein the frame is positioned on the support through the binding layer, wherein the binding layer includes a blackening agent for absorbance.

According to various embodiments, an apparatus for hybridization can include a substrate, and a hybridization chamber including a detection frame, a cover, and a portion of the substrate, wherein the cover includes a cavity to disperse a hybridization fluid over the portion of the substrate, wherein the detection frame is positioned on the substrate, and the substrate is configured for SPR detection. According to various embodiments, an apparatus for hybridization can include a housing including a cover, a substrate including a microarray, and a hybridization chamber including a frame and a portion of the substrate and wherein the cover includes a cavity for dispersing a hybridization fluid over the microarray, wherein the cover is mated with the frame to seal the hybridization chamber, wherein the cover includes an inlet port, and wherein the substrate is configured for SPR detection.

According to various embodiments, a method for hybridizing can include providing a housing including a cover, providing a substrate including a microarray, providing a hybridization chamber including a frame and a portion of the substrate, dispersing a hybridization fluid over the microarray, and detecting hybridization by SPR.

It is to be understood that both the foregoing general description and the following description of various embodiments are various and explanatory only and are not restrictive.

### BRIEF DESCRIPTION OF THE DRAWINGS

The accompanying drawings, which are incorporated in and constitute a part of this specification, illustrate various embodiments. In the drawings,

FIGS. 1A-1C illustrate various embodiments of proximate detection and hybridization frames;

FIGS. 2A-2C illustrate various embodiments of the cover;

FIGS. 3-4 illustrate various embodiments of the apparatus assembled and disassembled;

FIGS. 5A-5D illustrate various embodiments of the inlet port and elevated portion;

FIGS. 6-11 illustrate various embodiments of the apparatus assembled and disassembled;

FIG. 12 illustrates various embodiment of the substrate and the detection frame with cover slip for detection;

FIG. 13 illustrates various embodiment of the substrate and the detection frame with a plurality of barriers forming an array of chambers and microarrays;

FIG. 14 illustrates various embodiment of the substrate and the detection frame with cover for SPR detection;

FIG. 15 illustrates various embodiment of the cover with an inlet port and an outlet port for hybridization fluid flow-through;

FIG. 16 illustrates various embodiment of the cover with an inlet port and an outlet port;

FIGS. 17A-17F illustrate various embodiments of the substrate magnified to show attachment of the detection frame to the support, adhesive layer, binding layer and the micro-array;

FIG. 18 illustrates various embodiments of the cap; and

FIG. 19 illustrates various embodiments of coupling for SPR.

### DESCRIPTION OF VARIOUS EMBODIMENTS

Reference will now be made to various embodiments, examples of which are illustrated in the accompanying drawings. Wherever possible, the same reference numbers are used in the drawings and the description to refer to the same or like parts.

The term “hybridization” as used herein refers to the process of forming a duplex between two members of specific binding pair. The specific binding pair is frequently complementary or partially complementary strands of a polynucleotide. It will be understood my those skilled in the art of molecular biology that the term “polynucleotide” as used herein includes analogs of naturally occurring polynucleotides and does not covey any limitation of the length of the polynucleotide. One of the polynucleotide strands may be immobilized on a solid substrate.

Polynucleotide strands used for hybridization can be labeled with a detectable marker to as facilitate the detection of duplexes. Examples of detectable markers can include, but are not limited to fluorescent dye, radioisotopes, enzyme, or other markers. According to various embodiments, detection can be provided by a CCD camera that detects the detectable markers. Polynucleotide strands used for hybridization can be non-labeled. Hybridization of non-labeled binding pairs can be detected by surface plasmon resonance (SPR).

Hybridization can be used for a variety of purposes, including understanding the structure-activity relationship between different materials, detecting and screening single nucleotide polymorphisms (SNPs), and sequencing an unknown material. The term “specific binding pair” refers to a pair of molecules that bind to one another with a specificity that is detectable above background levels of non-specific molecular interactions. Examples of specific binding pairs can include, but are not limited to antibody-antigen (or hapten) pairs, ligand-receptor pairs, biotin-avidin pairs, polynucleotides with complementary base pairs, nucleic acid binding proteins and cognate nucleic sequences, members of multi-protein complexes, and the like. Each specific binding pair can include two members, or additional compounds can specifically bind to either member of a given specific binding pair.

According to various embodiments, the apparatus for hybridization can provide a hybridization chamber with polynucleotides in the liquid phase or bound on a substrate. For illustrative purposes, the figures describe polynucleotides bound on a substrate to form a microarray. Microarrays can have densities of 4 binding sites per square millimeter or up to 10⁴ binding sites per square millimeter. Binding sites can be positioned on the substrate by pin spotting, ink-jetting, photo-lithography, and other methods known in the art of high density deposition. The features described in the figures can be easily applied to an apparatus for hybridization with nucleotides in the liquid phase.

According to various embodiments, “SPR” as used herein refer to a surface-sensitive, optical detection method that can be used to monitor hybridization and other protein-nucleic acid binding. SPR can be sensitive to the thickness and index of refraction of material at the interface between a free electron metal (e.g., gold, silver, copper, cadmium, aluminum) and a bulk medium, such as air or water. The microarray can be deposited on the metal substrate. SPR can use an evanescent wave generated by a light source that is substantially monochromatic, incoherent, near-infrared light source, for example a laser beam or light-emitting diode (LED). The wave can be linearly polarized parallel to the plane of incidence to impinge onto the metal. The metal can be coated onto a support, including glass or plastic such as polycarbonate, to form a thin film portion of the substrate. The substrate can be constructed entirely out of metal.

According to various embodiments, as illustrated in FIG. 19, the substrate can be coupled to a prism (Kretschmann Prism Coupling), or the substrate can be coupled a grating (Grating Coupling). SPR can be observed as the reduction in light intensity reflected from the prism-metal interface (Kretschmann Prism Coupling) or from the grating-metal interface (Grating Coupling) when, for a correct combination of refractive indices, incident light wavelength, and angle of incidence, as indicated by θ in FIG. 19, photons from an incident light beam enter into surface plasmon modes within the metal and are absorbed. According to various embodiments, the angle of incidence for greatest photon absorption, and hence for least reflected light intensity, can be determined. Hybridization on the metal surface of the substrate can change the index of refraction, thereby shifting the angle of incidence for least reflected light intensity. The shift in the angle of incidence can be used to detect the accretion or reduction of materials on the metal surface in contact with the hybridization fluid.

According to various embodiments, the binding of specific binding pairs can increase the thickness of the microarray such that the microarray can provide a shift in the angle of incidence that can indicate the presence of binding. By monitoring either the position of the angle of incidence or the reflectivity at a fixed angle near the angle of incidence, the presence or absence of binding at a binding site can be detected. SPR is relatively fast and can be performed without labeling. Detection can be provided by a CCD camera at a fixed angle. The SPR image arises from variations in the reflected light intensity from different binding sites. The hybridization chamber with a transparent cover can be used for detection with SPR.

According to various embodiments, grating-coupled SPR can be observed by a CCD camera detector from a microarray with a surface area of at least 1.0 cm by 1.0 cm. The CCD camera can provide simultaneous detection of the entire matrix of binding sites on the microarray.

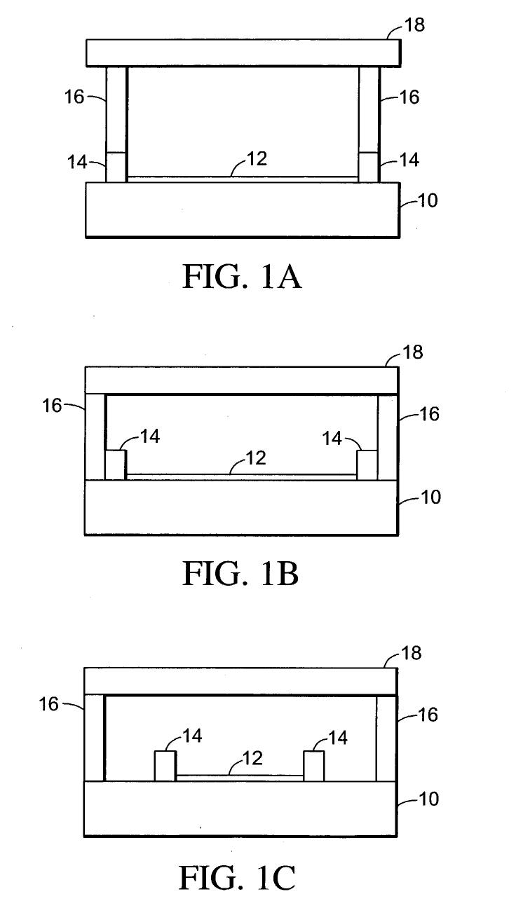

According to various embodiments, FIGS. 1A-1C illustrate an apparatus for hybridization showing the relative position of the detection frame and hybridization frame. These embodiments can relate to a housing or an instrument. According to various embodiments, the instrument can provide a lid and a holder configured like jaws that can be held together to form the hybridization chamber. In FIGS. 1A-1C, the complete housing or instrument is not shown. Detection frame 14 can be positioned on substrate 10 bounding micro-array 12. Hybridization frame 16 can be positioned proximate to said detection frame such that it can form a seal with cover/lid 18 (cover in the housing embodiment and lid in the instrument embodiment). According to various embodiments, hybridization frame 16 can be compressible to better form such seal. Compressible materials are known in the material science arts. According to various embodiments, the hybridization frame and/or the detection frame can be constructed of at least one elastomeric material chosen from Silicone Rubber, FDA approved Silicone Rubber, EPDM Rubber, Neoprame (CR) Rubber, SBR Rubber, Nitrile (NBR) Rubber, Butyl Rubber, Hypalon (CSM) Rubber, Polyurethane (PU) Rubber, Viton Rubber, and polydimethylsiloxane (Slygard™ elastomer by Dow Corning).

The term “frame” as used herein refers to gaskets, rings, or seals. The frame can be angular or circular in overall shape and/or cross-section. The frame can be constructed of compressible, elastomeric material. The frame does not include any internal means of channeling flow such as ports or means of mixing such channeled flow such as a reaction recess in the frame. The frame does not permit leaching of contaminants into the hybridization chamber. The frame is unitary and does not include multiple members. The frame is not separated into multiple frames such as grid plates. The term “detection frame” as used herein refers to the frame that forms the detection chamber and adheres on the substrate. The term “hybridization frame” as used herein refers to frame that forms the hybridization chamber either with the detection frame by being positioned on the detection frame or outside the detection frame by being positioned adjacent or near the detection frame, or without the detection frame.

The term “proximate” as used herein refers to the location of the detection and hybridization frames, including positioned on each other such that they are one on top of the other with each one having the same width or either one being wider, adjacent to each other such that they are contact each other on the side with each one having the same center or either one being off center, or near each other such that they do not contact each other but are a short distance apart with either one surrounding the other.

According to various embodiments, FIG. 1A illustrates hybridization frame 16 can be positioned on detection frame 14. According to various embodiments, detection frame 14 can be harder than hybridization frame 16 to provide more stability during detection. Hybridization frame 16 can be compressed between detection frame 14 and cover/lid 18 to form a sealed hybridization chamber. FIG. 1B illustrates hybridization frame 16 can be positioned adjacent to detection frame 14. Hybridization frame 16 can be compressed between substrate 10 and cover/lid 18 to form a sealed hybridization chamber. FIG. 1C illustrates hybridization frame 16 can be positioned near detection frame 14. Hybridization frame 16 can be compressed between substrate 10 and cover/lid 18 to form a sealed hybridization chamber. Although the detection and hybridization frames illustrated in FIGS. 1A-1C are rectilinear, detection and hybridization frames can be any shape including circular, elliptical, and combinations thereof. As described below, the hybridization chamber can include overlapping hybridization volume and detection volume.

According to various embodiments, the detection frame and hybridization frame can be replaced with one frame that forms both the hybridization chamber and the detection chamber. In the SPR detection embodiment, the hybridization chamber can also be the detection chamber. It is clear to one skilled in the art that the features described in the two-frame embodiments can similarly benefit one-frame embodiments.

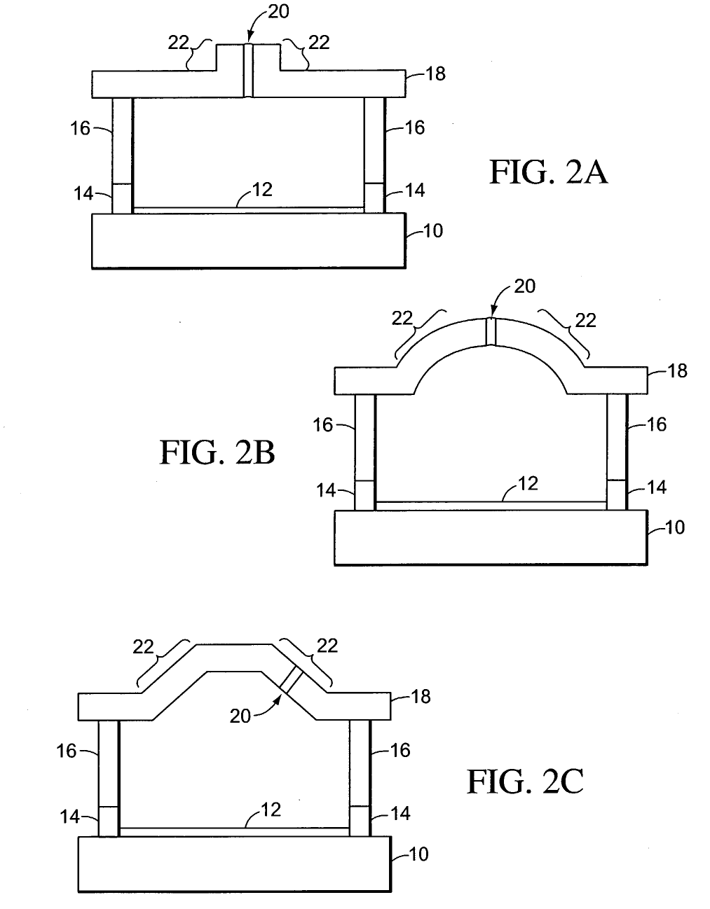

According to various embodiments, the apparatus for hybridization can include a housing or an instrument. As illustrated in FIGS. 1A-1C, the apparatus for hybridization can relate to either a housing or instrument without a housing. In a cartridge, the cover and base of the housing can operate in a similar fashion as the lid and holder of an instrument. According to various embodiments, FIGS. 2-11 and FIGS. 14-16 can relate to a housing. According to various embodiments, a housing can be positioned within an instrument for processing (including filling, binding reactions, washing, drying, etc.) and benefit from the features illustrated in FIGS. 2-11 and FIGS. 14-16. The housing can be removed from the instrument for detection or detection can take place in the instrument.

According to various embodiments, FIGS. 2A-2C illustrate an apparatus for hybridization using a housing having different covers. FIGS. 2A-2C illustrate detection frame 14 can be positioned on substrate 10 bounding microarray 12 and hybridization frame 16 can be positioned on detection frame 14. Hybridization frame 16 can be compressible so that it forms a seal with cover 18 to enclose the hybridization chamber. According to various embodiments, the detection frame and hybridization frame can be proximate to each other, as described above. FIGS. 2A-2C illustrate that cover 18 can have an elevated portion 22 and an inlet port 20. As illustrated in FIG. 2A, elevated portion 22 can project from the upper surface of cover 18 such that cover 18 is substantially parallel to microarray 12 on substrate 10. As illustrated in FIGS. 2B and 2C, elevated portion 22 can be a portion of said cover and can be domed or vaulted such that cover 18 is not substantially parallel to microarray 12 on substrate 10. FIG. 2B illustrates an elevated portion 22 which can have a hemispherical dome. FIG. 2C illustrates an elevated portion 22 which can have a rectilinear or angled vault. Any other variation or combination of a dome or a vault can be used to increase the hybridization volume of the hybridization chamber. As illustrated in FIG. 2C, port 20 can be moved to the side of the elevated portion 22. The port does not have to be centered and can be anywhere on the elevated portion or other portion of the cover. The elevated portion can be a separate component other than the cover that attaches to the cover.

According to various embodiments, the hybridization chamber provides a hybridization volume less than 300 microliters. The amount of hybridization fluid can be 1.0 to 50 microliters.

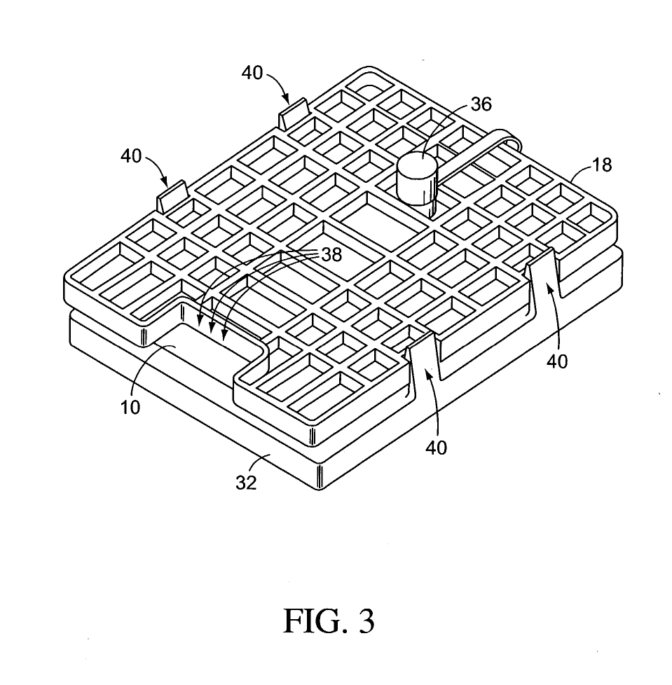

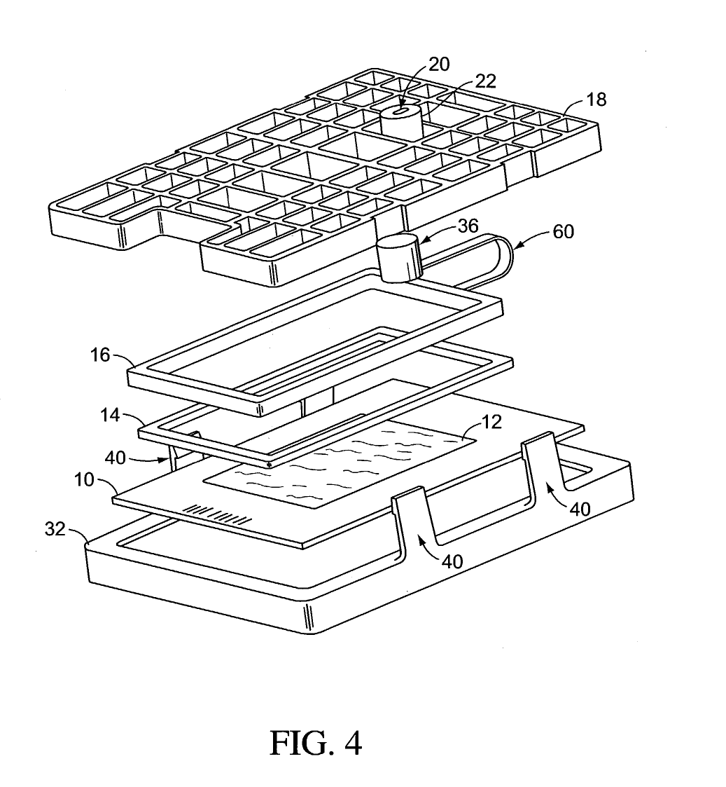

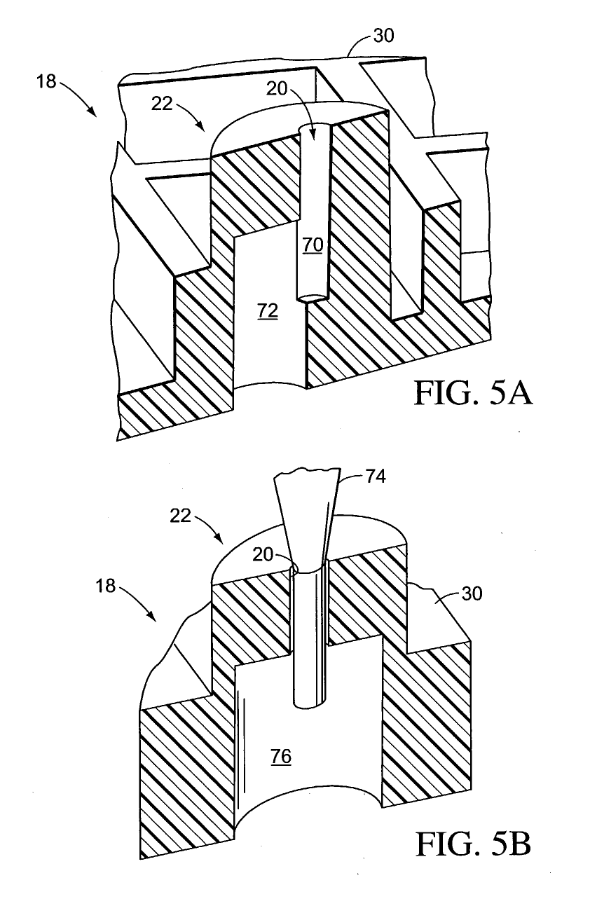

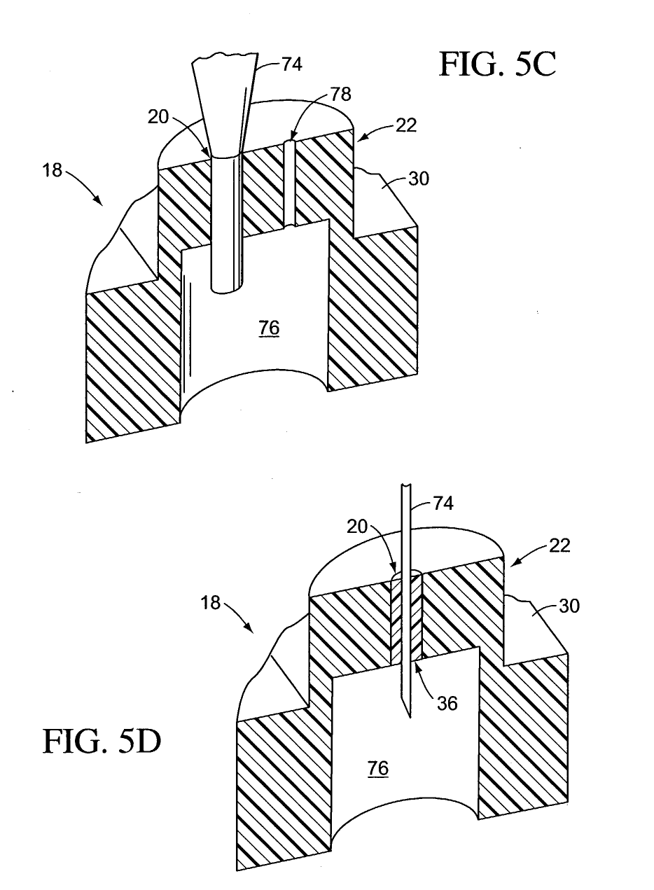

According to various embodiments, FIGS. 2-11 and FIGS. 14-16 illustrate an apparatus for hybridization including a housing with cover and base. According to various embodiments, a housing can be assembled in a locked position and disassembled in an unlocked position. The housing can include a cover which itself can include a top and a compression plate. The compression plate can include an elevated portion, inlet port, and an exhaust port to allow the gas displaced by the hybridization liquid to escape to the atmosphere. The inlet port and exhaust port can be blocked to prevent hybridization fluid from escaping from the hybridization chamber by a cap. According to various embodiments, the cap can be a sealing tape on the top of inlet port, can enclose the elevated portion (e.g. FIGS. 3 and 4), or can plug the inlet port (e.g. FIG. 5D). The cover and base in a locked position can form a window that exposes a portion of the underlying substrate. A bar code can be positioned on the to substrate and can be accessed and/or read through the window. The top can provide an opening to allow the elevated portion of the compression plate to protrude. The top and base can include a releasably mating mechanism. The compression plate can have a raised portion on the opposite side to elevated portion to engage hybridization frame. Splitting the cover into a top and compression plate can provide a releasably mating mechanism that can be moved in a locked position by pressing down, sliding, rotating, and/or flipping the top. Pressing down can engage the compression plate on hybridization frame. Sliding, rotating, and/or flipping positions the top to the locked position without moving the compression plate. The top can be released by sliding, rotating, and/or flipping it in the reverse direction. According to various embodiments, methods for hybridization include providing the apparatus and releasably mating the cover and base according these actions.

According to various embodiments, a bar code can be positioned on the substrate. The term “bar code” refers to any marking that can identify the substrate, including identifying the microarray and/or hybridization. The bar code can be one-dimensional (e.g. bars), two-dimensional (e.g. dot matrices), or three-dimensional (e.g. holograms). Positioning a bar code on the substrate can include positioning an adhesive label with a bar code printed on the label, positioning multi-layered opaque coatings of contrasting colors, or marking the substrate directly, as is described below. According to various embodiments, a bar code can be positioned on the housing or frames.

According to various embodiments, a bar code can be positioned directly on a metal substrate by marking. A bar code can be marked on the metal substrate with a light beam from a laser or LED. The bar code can be marked to a depth within the substrate so that it can be read with a reader apparatus which can read refracted laser light from the substrate after the substrate is exposed to a light beam from the reader apparatus. The bar code can be marked onto the substrate to a depth into the substrate such that incident light refracted from the substrate can show the bar code as being black on a white background or white on a black background. According to various embodiments, the bar code can be positioned in one step or a plurality of steps. The substrate surface can be modified by ablating metal residue on the surface. Ablating can provide a uniform surface for positioning the bar code and can establish a consistent angle of refraction of the returned light pattern. The substrate surface can be marked with a bar code. The marked bar code can be read by exposing the bar code to a light beam from a reader apparatus. The reader can be positioned to determine whether the marked bar code provides sufficient contrast to its background to permit its being read by the reader apparatus. According to various embodiments, the bar code can be marked on the substrate to a depth between 1.27×10⁻⁵ meters and 2.54×10⁻⁵ meters.

According to various embodiments, the housing, including the cover and base can be manufactured from injection molding plastic. Injection molding can reduce the cost of producing the housing and can provide a disposable container that can be discarded after hybridization processing. The housing can be composed of acrylonitrile-butadiene-styrene plastic, polyurethane, polyvinylchloride, polycarbonate, polyethylene, TEFLON™, polystyrene, KALREZ™, or other materials known in the art of consumables manufacturing.

The term “releasably mated” or “releasably mating” as used herein refers to mating the cover and base such that they can be in a locked position during hybridization and then released to provide access to the substrate for detection. The base and cover are releasably mated by the releasably mating mechanism. Various embodiments of the releasably mating mechanism are described herein. According to various embodiments, the locked position can be subjected to several types agitation under which the cover and base remain mated. These types of agitation include tilting the housing, rolling the housing, moving the housing back and forth, moving the housing in a circular pattern, and rotating the housing about an axis.

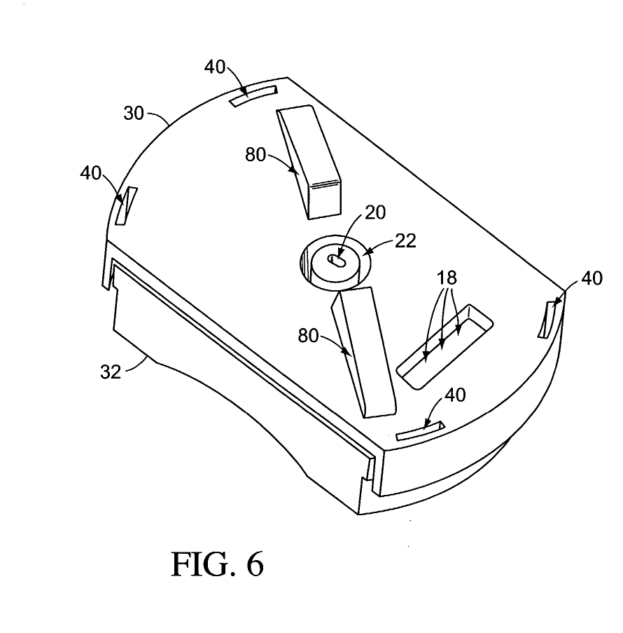

FIG. 3 illustrates an embodiment of a housing assembled and in a locked position. FIG. 4 illustrates the housing of FIG. 3 disassembled. The housing includes cover 18 and base 32 held in the locked position by releasably mating mechanism 40. The releasably mating mechanism 40 can move the cover 18 to the locked position by pressing down and clamping. The cover 18 can be released by unclamping releasably mating mechanism 40 and pulling up. An indentation in cover 18 can form window 38. A bar code (not shown) positioned on substrate 10 can be read through window 38. Cap 36 can block hybridization fluid from escaping from the hybridization chamber. FIG. 6 illustrates the housing disassembled. Cap 36 can be connected to hybridization frame 16 via tether 60. Both the cap 36 and hybridization frame 16 can be removed to provide access to the substrate 10 for detection.

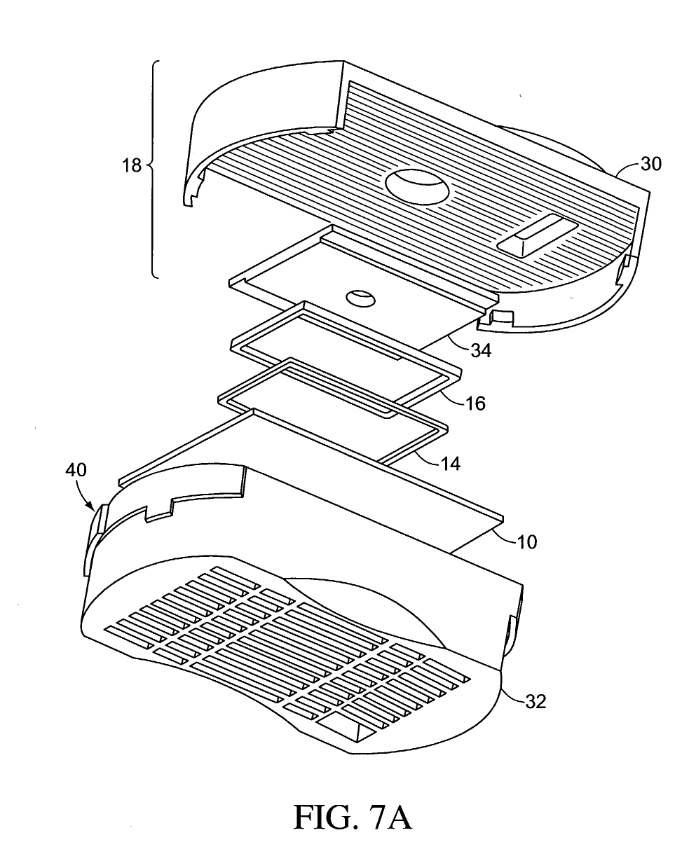

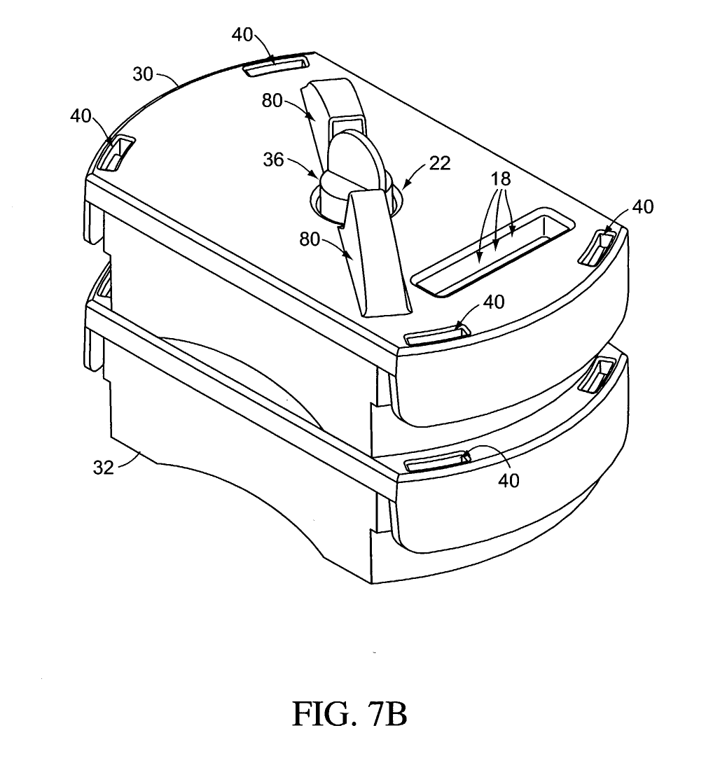

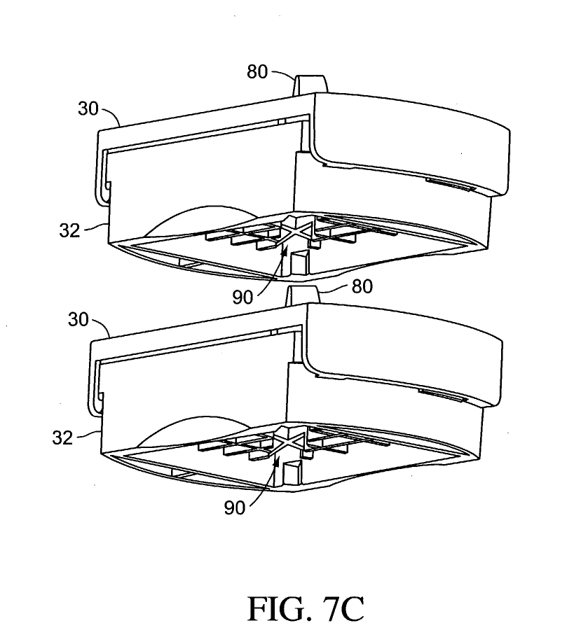

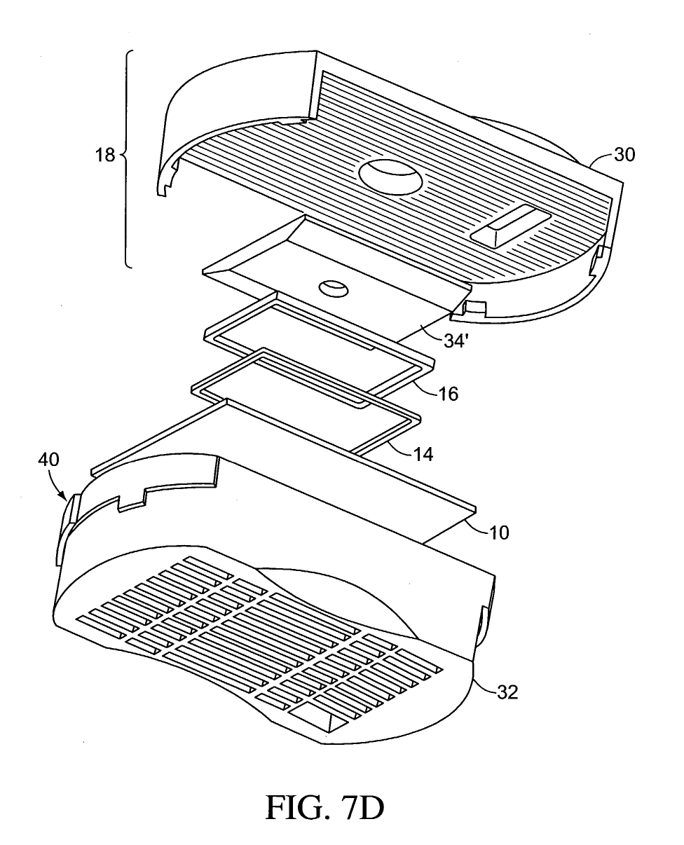

According to various embodiments, FIG. 6 illustrates a housing assembled and in a locked position. FIG. 7A illustrates the housing of FIG. 6 disassembled. The housing can include base 32 and cover 18. Cover 18 can include top 30 and compression plate 34. Top 30 and base 32 can include releasably mating mechanism 40. Top 30 can include handle 80 for manually handling top 30, and window 38 for accessing the bar code (not shown) positioned on substrate 10. Top 30 can have an opening to allow the elevated portion 22 of compression plate 34 to protrude. Releasably mating mechanism 40 can be moved to a locked position by rotating top 30. Splitting the cover 18 into top 30 and compression plate 34 can provide for a releasably mating mechanism 40 that can be moved in a locked position by rotating the top without rotating the compression plate 34. Rotating top 30 can push compression plate 34 down to engage hybridization frame 16. Top 34 can be released by rotating in the reverse direction and pulling up. The underside of base 32 can include an indentation that provides stacking of one housing on top of another. According to various embodiments, FIG. 7C illustrates that handles 80, elevated portion 22, and cap 36 can fit into indentation 90 to permit stacking of two or more housings. FIG. 7B illustrates the housings stacked on top of each other. According to various embodiments, compression plate 34 can have a non-uniform thickness to provide compression of hybridization frame 16. FIG. 7D illustrates compression plate 34′ that has non-uniform thickness.

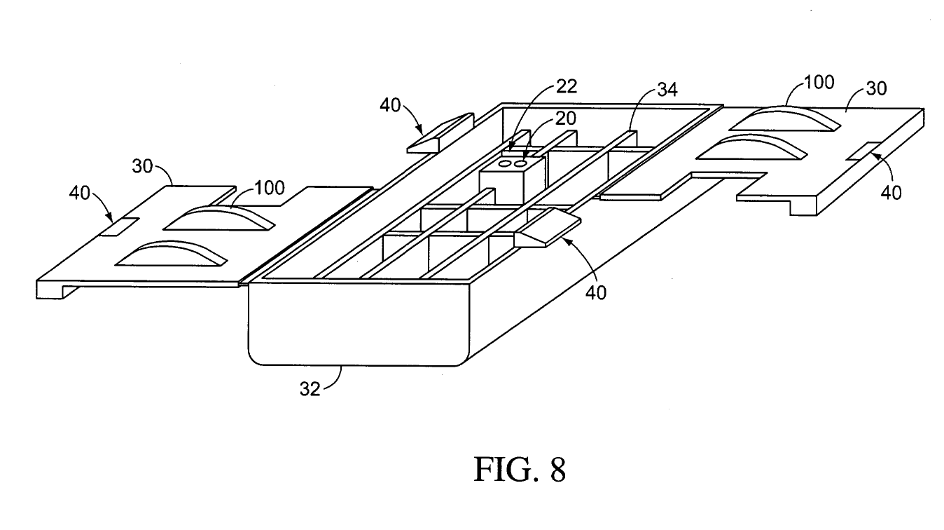

According to various embodiments, FIG. 8 illustrates a housing that is assembled but not in the locked position. Compression plate 34 can be positioned in base 32. Top 30 can be hinged on base 32. Top 30 can be divided into two portions that are hinged on opposite sides of base 32. According to various embodiments, the top 30 can be hinged on one side of base 32 or the top can be divided into more than two portions. Top 30 can include ribs 100 to compress compression plate 34 when the cover is moved to the locked position. The two portions of top 30 can form an opening so that the elevated portion 22 of compression plate 34 can protrude through top 30 when the cover is in a locked position. Top 30 and base 32 can include releasably mating mechanism 40. Releasably mating mechanism 40 can clamp top 30 to base 32 when the top 30 is flipped along the hinged portion into the locked position. Clamping top 30 to base 32 can force ribs 100 to push compression plate 34 down to engage the hybridization frame (not shown). Top 30 can be released by unclamping releasably mating mechanism 40.

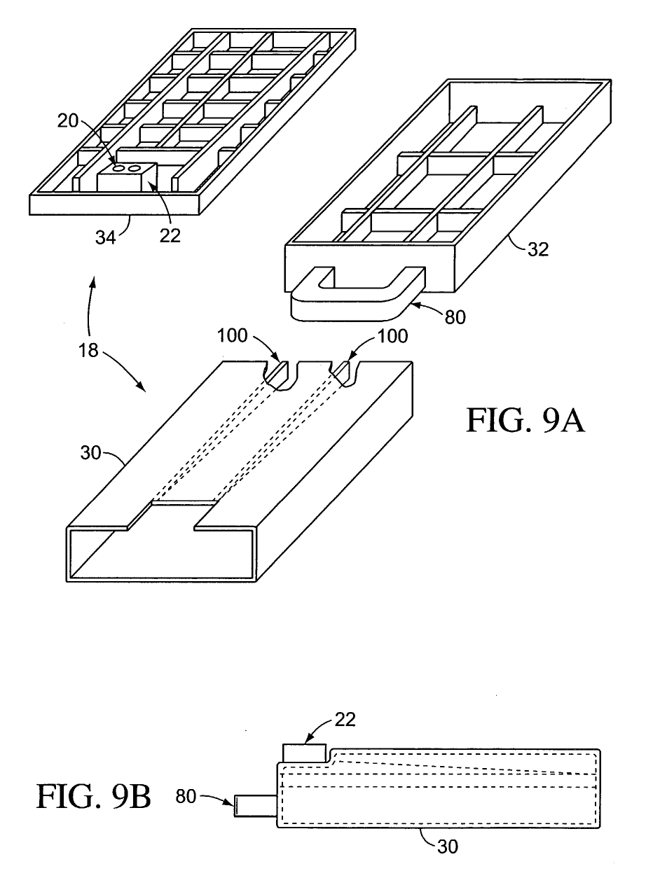

According to various embodiments, FIGS. 9A-9B illustrate a housing that is unassembled and assembled, respectively. FIG. 9A illustrates the housing unassembled with cover 18 including top 30 and compression plate 34. Compression plate 34 can include elevated portion 22 and inlet port 20. Top 30 can include five sides similar to a “match-box” with a closed end. Base 32 can include handle 80 for pushing and pulling base 32 into and out of top 30. Top 30 can include ribs 100. Compression plate 34 can include sloped ribs to compress compression plate 34 when it is positioned in base 32 and base 32 is pushed into top 30 to the locked position. FIG. 9B illustrates a side view of the assembled housing. The broken lines illustrate the interior of the housing, including base 32, compression plate 34, and ribs 100. Top 30 can include an opening that allows elevated portion 22 to protrude above top 30. Handle 80 can protrude from the open end of top 30. Ribs 100 and the sloped ribs of compression plate 34 can provide the releasably mating mechanism and compress compression plate 34. Pushing base 32 into top 30 can force ribs 100 to push compression plate 34 down to engage the hybridization frame (not shown). Base 34 can be released by pulling handle 80 horizontally away from top 30.

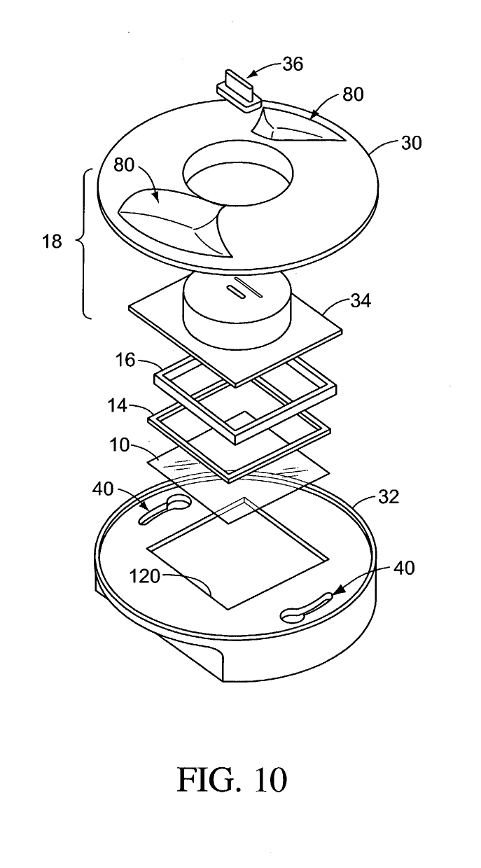

According to various embodiments, FIG. 10 illustrates a housing that is unassembled and assembled. FIG. 10 illustrates the housing unassembled with cover 18 which can include top 30 and compression plate 34. Top 30 can include handles 80 for rotating top 30 to the locked position. Cap 36 can block hybridization fluid from escaping from the hybridization chamber through inlet port 20 in elevated portion 22 of compression plate 34. Top 30 can include an opening to allow elevated portion 22 of compression plate 34 to protrude above top 30. Base 32 and top 30 can include releasably mating mechanism 40. Releasably mating mechanism 40 can include prongs that move along a guide when the top 30 is rotated to move the top 30 to a locked position. Splitting the cover 18 into a top 30 and compression plate 34 can provide for releasably mating mechanism 40 that is moved in a locked position by rotating top 30 without rotating compression plate 34. Rotating top 30 can push compression plate 34 down to engage hybridization frame 16. Top 34 can be released by rotating in the reverse direction and pulling up. Base 32 can include window 120 that provides access to the underside of substrate 10. A bar code can be positioned to the underside of substrate 10 can be accessed through window 120.

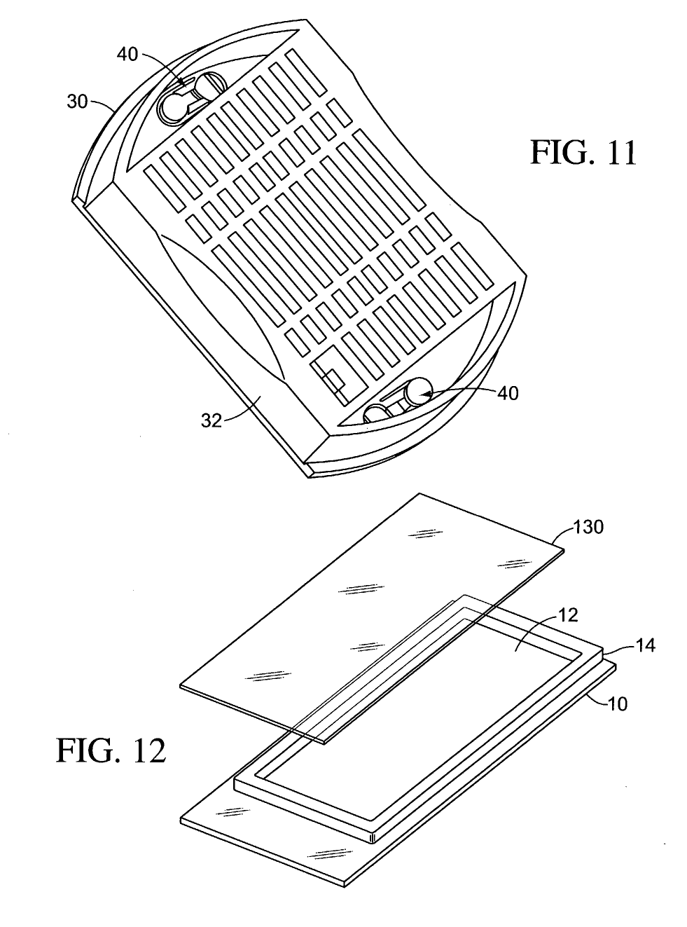

According to various embodiments, FIG. 11 illustrates a bottom view of an assembled housing similar to the housing illustrated in FIG. 6, but with a different releasably mating mechanism. Base 32 can include openings that provide visual access to substrate 10. Releasably mating mechanism 40 can maintain top 30 and base 32 in the locked position. FIG. 10 illustrates releasably mating mechanism 40 that can include prongs that can move along a guide when top 30 is rotated to move the top 30 in a locked position. FIG. 11 illustrates releasably mating mechanism 40 that can include prongs that can move along a guide when the top 30 is rotated to move the top 30 in a locked position, where the guide can include flexible tabs which provide some flexibility between top 30 and base 32 during insertion of the prong into the guide and/or during rotation.

According to various embodiments, the housing can relate to batch-processing hybridization or continuous-processing hybridization. It is understood by one skilled in the art, that features of batch-processing hybridization can be applied to continuous-processing hybridization by adding an outlet port. It is also understood by one skilled in the art, that features of continuous-processing hybridization can be applied to batch-processing hybridization by eliminating the outlet port. It is also understood by one skilled in the art, that in a combination of batch-processing hybridization and continuous-processing hybridization the features of both can be applied.

According to various embodiments, FIGS. 14-16 illustrate an apparatus for hybridization including a housing where the cover mates directly with the base, which is the substrate. It is understood by one skilled in the art, that features of housings where the substrate is positioned in the base can be applied to housings where the substrate is the base. It is also understood that features of housings where the substrate is the base can be applied to housings where the substrate is positioned in the base.

According to various embodiments, FIG. 14 illustrates a housing including a cover 18 and a substrate 10, wherein the substrate is the base. Cover 18 can include window 150. Window 150 can be a portion of cover 18 or it can be transparent material that forms to the surface of cover 18. Cover 18 includes inlet port 20 and outlet port 160. The detection frame 14 (which is also the hybridization frame) can be positioned between cover 18 and substrate 10. Substrate 10 can be adapted for SPR. Substrate 10 can include a free electron metal. Substrate 10 can be constructed of metal or substrate 10 can include of a support coated with a thin metal film. According to various embodiments, as illustrated in FIG. 19(a), substrate 10 can be coupled to grating 190. According to various embodiments, as illustrated in FIG. 19(b), substrate 10 can be coupled to prism 192. FIG. 19 illustrates angle of incidence θ and the arrow in the grating or prism illustrates the direction of propagation for the surface plasmon. According to various embodiments, as illustrated in FIG. 14, a microarray, a grating or a prism in the hybridization chamber can be aligned with window 150 to provide access for light from a light source and toward a detector.

According to various embodiments, FIG. 16 illustrates the bottom of cover 18. Cover 18 can include cavity 72 which can disperse or collect the hybridization fluid from inlet port 20 or outlet port 160, respectively. According to various embodiments, cover 18 can include an inlet port 20 with cavity 72. Cavity 72 can include a uniform expansion or graduated expanding sections. Cavity 72 can disperse the hybridization fluid over the portion of the substrate that includes the microarray. Dispersing the hybridization fluid can provide uniform contact between the hybridization fluid and the microarray. Cover 18 can be mated to detection frame 14 with an adhesive that is compatible with both detection frame 14 and cover 18. The adhesive provide either releasable mating or permanent mating.

According to various embodiments, FIG. 15 illustrates a housing where the substrate can be the base. According to various embodiments, any base described above can be used with the cover shown in FIG. 15. FIG. 15 illustrates that cover 18 can include top 30 and compression plate 34. Top 30 can include two elevated portions 22. One elevated portion 22 includes inlet port 20 and the other elevated portion includes outlet port 160. Compression plate 34 compresses hybridization frame 16 on detection frame 14 which can be attached to substrate 10 when top 30 contacts substrate 10. FIG. 16 illustrates a bottom view of top 30. Top 30 can include a dispensing cavity 72 adjacent to inlet port 20. Dispensing cavity 72 can be positioned on the inside of hybridization frame 16 and can provide a telescoping width to disperse the hybridization liquid over the portion of substrate 10 within the hybridization chamber. There can be a similar structure adjacent to outlet port 160 to collect the hybridization liquid from the hybridization chamber.

According to various embodiments, the releasably mating mechanism can include of spring tabs, prongs and guides, flexible tabs that move the cover to the locked position once the desired compression is achieved. In addition to these, any variety of releasable fastener known in the art of packaging can be implemented as a releasably mating mechanism.

It is understood to one skilled in the art that combining or selecting features of various embodiments of the housing can apply the housing to the particular application and processing environment for the hybridization. As such many permutations of the features described above are contemplated in this description.

According to various embodiments, FIGS. 5A-5D illustrate the inlet port and elevated portion of the cover. FIGS. 5A-5D illustrate an elevated portion similar to FIG. 2A where the elevated portion can project from the upper surface of the cover. The features described in conjunction with FIGS. 5A-5D can also apply to elevated portions illustrated in FIGS. 2B and 2C where the elevated portion can be a portion of the cover. The features described in conjunction with FIGS. 5A-5D can apply to elevated portions that are separate components from the cover. The features described in conjunction with FIGS. 5A-5D can apply to inlet ports directly in the cover without elevated portions. The FIG. 5A illustrates a cross-section of elevated portion 22 which includes inlet port 20, filling cavity 70 and dispensing cavity 72. Filling cavity 70 and dispensing cavity 72 can be in fluid contact with vertical overlap and slight horizontal overlap. The horizontal overlap can provide a stop for the filling device. The filling device can provide the hybridization fluid to the hybridization chamber but the stop can prevent the filling device from contacting the substrate below the dispensing cavity 72 and hybridization chamber.

The term “filling device” as used herein refers to any device for providing hybridization fluid to the hybridization chamber. Filling devices can include, but are not limited to a pipette, a syringe, a tube, and any other device known in the art of handling laboratory or manufacturing fluids. The filling device can be automated or manually operated.

FIG. 5B illustrates a cross-section of filling device 74 positioned into inlet port 20 of elevated portion 22. Inlet port 20 can be configured to restrain the filling device 74 to prevent the filling device 74 from contacting the substrate below. Inlet port 20 can be configured to provide an opening around filling device 74 to allow the gas displaced by the hybridization fluid to escape to the environment. With the filling device 74 restrained, the hybridization fluid can be dispensed into overhead cavity 76. The hybridization fluid fills only a portion of the hybridization volume leaving overhead cavity 76 clear. FIG. 5C illustrates a cross-section of filling device 74 positioned into inlet port 20 of elevated portion 22. Elevated portion 22 can include an exhaust port 78 to allow the gas displaced by the hybridization fluid to escape to the environment. The exhaust port 78 can be configured to have a small enough width such that no substantial amount of hybridization fluid escapes during hybridization or can be covered by cap 36 along with inlet port 20. FIG. 5D illustrates a cross-section of filling device 74 positioned in inlet port 20 of elevated portion 22. Inlet port 20 can be plugged by a plug or can be plugged by a portion of cap 36. Plugged inlet port 20 is configured to restrain filling device 74. The plug and/or cap can be constructed of a gas permeably material to allow the gas displaced by the hybridization fluid to escape while retaining any evaporating hybridization fluid during the hybridization protocol. Elevated portion 22 can provide depth for insertion of filling device 74 into overhead cavity 76 to prevent the filing device 74 from contacting the substrate below. The length and/or the width of inlet port 20 can be configured to prevent filling device 74 from contacting the substrate. According to various embodiments, the other portions of the cover can include an exhaust port to allow the gas displaced by the hybridization fluid to escape while the inlet port is occupied by the filling device.

According to various embodiments, the inlet port can be configured to be cylindrical or conical. The inlet port can be tapered holes to receive a filling device tip or the tapered tip of a filling device from an automated liquid delivery apparatus. The inlet port (or outlet port) can be threaded to accept liquid fittings, such as valves, Luers, barbs, o-rings, etc. for automated filing. The inlet port can be sealed with a septum, a tapered plug, threaded plug, or similar device that can be part of or separate from the cap.

According to various embodiments, adding the hybridization fluid includes taking the substrate through several wash and reagent addition steps and agitation as known in the art of hybridization. The substrate can then be removed from the housing manually or robotically. The detection chamber can be filled with overlay fluid to facilitate detection and remove any bubbles from the detection chamber that can interfere with detection. A cover slip or overlay is positioned on the detection frame to provide a flat optical surface for detection.

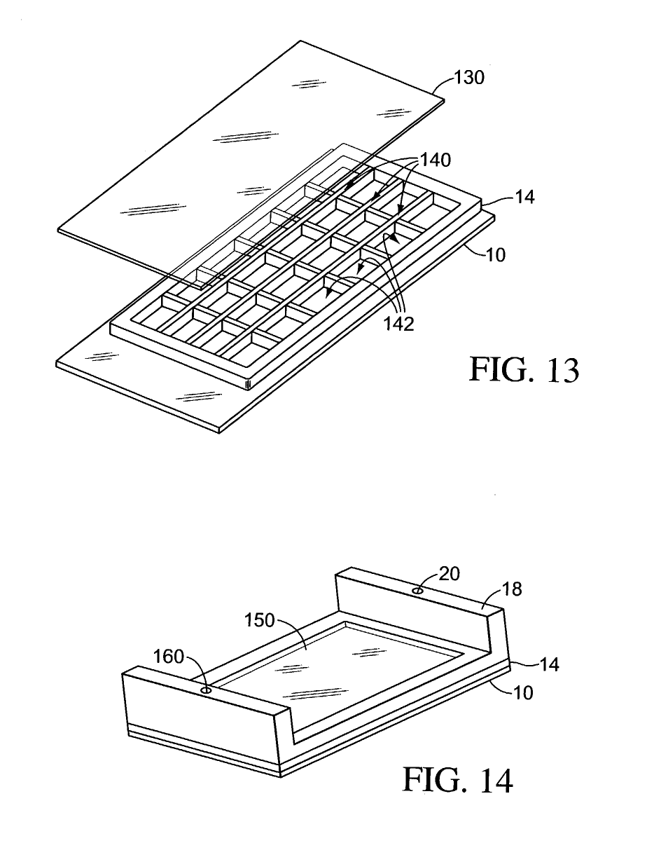

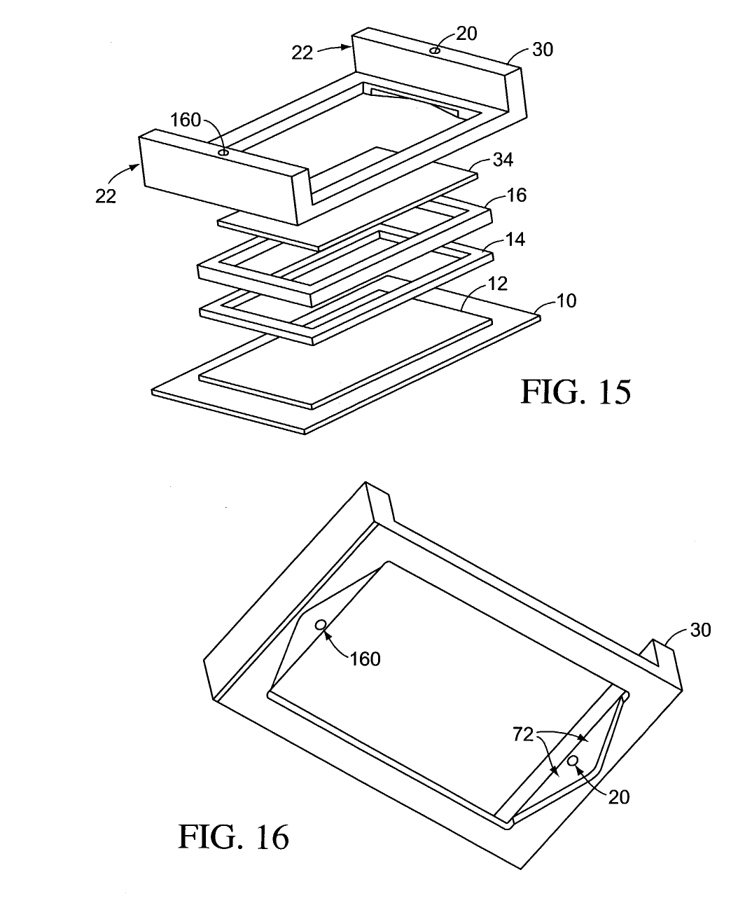

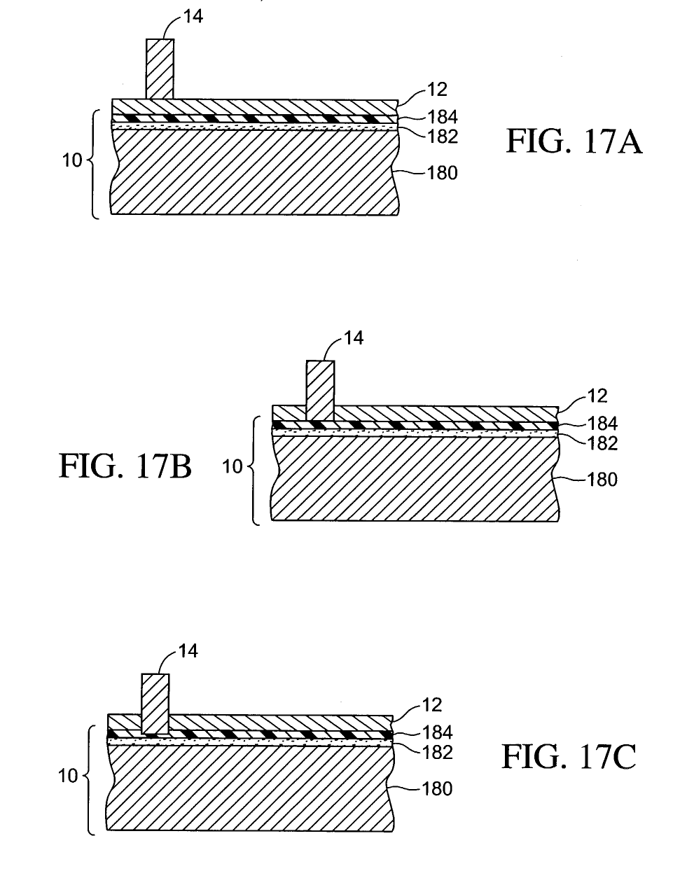

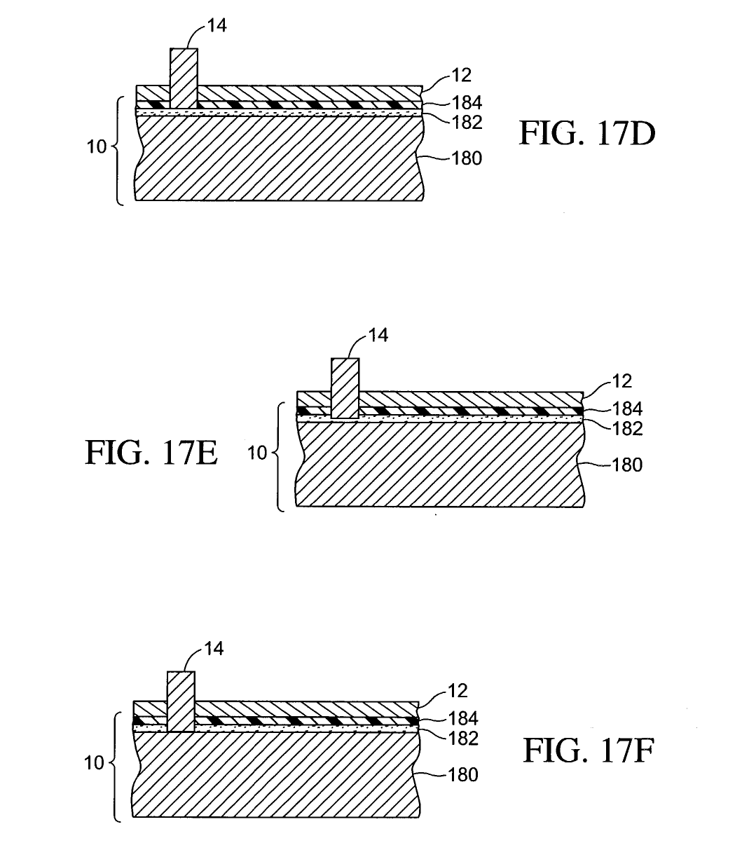

According to various embodiments, FIGS. 12 and 13 illustrate the substrate and detection chamber. FIG. 12 illustrates substrate 10 with detection frame 14 bounding microarray 12. Microarray 12 can be unitary as in FIG. 12 or divided into sections. Detection frame 14 can be made of harder material than the hybridization frame material. The detection frame 14 configured to maintain cover slip 130 substantially parallel to microarray 12 enclosing a detection volume of hybridization fluid in the detection chamber. Cover slip 130 can be transparent for detection and can be constructed of glass or plastic. In one embodiment, the detection chamber can hold 2.0 to 3.0 milliliters of detection volume. FIG. 13 illustrates substrate 10 with detection frame 14 bounding an array of microarrays 142 separated by barriers 140. Barriers 140 divide the detection chamber into an array of chambers such that positioning a cover slip on detection frame 14 provides an array of fluidically isolated chambers which can be interrogated individually by a detector.

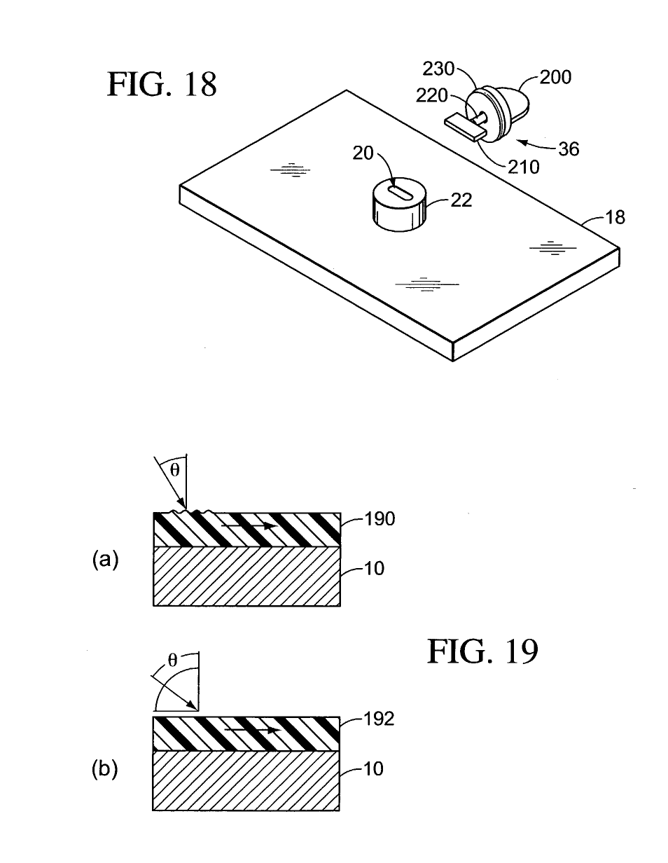

According to various embodiments, FIGS. 18A-18F illustrates a cross-section of the substrate and detection frame. FIGS. 18A-18F illustrate that substrate 10 can include support 180, adhesive layer 182, and binding layer 184. The microarray 12 binds to binding layer 184. Binding layer 184 can be NYLON, cellulose, nitrocellulose, gel, polymeric, or other porous membrane known in the art of polymer chemistry. Binding layer 184 can sprayed on, laminated on, deposited on via chemical vapor deposition, or deposited on via electrostatic deposition on the substrate support 180 and adhesive layer 182. The support 180 can be non-absorbent. The support 180 can be glass, fused silica, silicon, plastic, metal, ceramic, or polymeric. The adhesive layer 182 can be hydrophobic to seal the hybridization chamber, such as a pressure sensitive acrylic adhesive. Detection frame 14 can be adhered to the microarray, any one of the layers in substrate 10, or the interface of those layers. Detection frame 14 can be adhered to each of these via its own adhesive or by using the adhesive properties of these layers. FIG. 18A illustrates detection from 14 adhered to microarray 12. FIG. 18B illustrates detection frame 14 adhered to the interface of binding layer 184 and micro-array 12. FIG. 18C illustrates detection frame 14 adhered to binding layer 184. FIG. 18D illustrates detection frame 14 adhered to the interface of binding layer 184 and adhesive layer 182. FIG. 18E illustrates detection frame 14 adhered to adhesive layer 182. FIG. 18F illustrates detection frame 14 adhered to the interface of adhesive layer 182 and support 180. Each of these adhesion contacts provides a seal for the bottom of the hybridization chamber. According to various embodiments, the detection frame 14 can be adhered to the adhesive layer 182 through binding layer 184. Support 180 can be coated with adhesive layer 182 and binding layer 184. The outline of detection frame 14 can be removed from the binding layer 184. Detection frame 14 can be positioned on the adhesive layer 182 where the binding layer has been removed.

According to various embodiments, binding layer 184 can be transparent or opaque. Binding layer 184 can be blackened to render it opaque and increase absorption of light during detection. Increasing absorption reduces light refracted by the binding layer 184 and reduces light that generates noise signals to the detector. Binding layer 184 can be blacked by adding carbon black into its composition. Binding 184 can include multiple layers of transparent or opaque quality.

According to various embodiments, a method for hybridizing can include providing a housing including a cover, providing a substrate including a microarray, providing a hybridization chamber including a frame and a portion of the substrate, dispersing a hybridization fluid over the microarray, and detecting hybridization by SPR. Providing the substrate can include coupling a grating to the substrate. Providing the substrate can include coupling a prism to the substrate.

According to various embodiments, a method for hybridizing can include providing a housing including a base and a cover, providing a substrate including a microarray, providing a hybridization chamber including a frame and a portion of the substrate, and releasably mating the cover to the base to seal the hybridization chamber and compress the frame. Releasably mating can include pressing down and sliding the cover to a locked position, pressing down and clamping the cover to a locked position, rotating the cover to a locked position, and sliding the base to a locked position. The cover can include a hinged portion so that releasably mating can include flipping and clamping the hinged portion to a locked position.

According to various embodiments, a method of hybridization can include providing a hybridization fluid to the hybridization volume, hybridizing the fluid with the microarray, releasing the cover and the base, removing the hybridization frame, positioning a cover slip over the detection frame to seal the detection volume, and detecting results from the hybridizing on the microarray. The method can include positioning the substrate within the base, positioning the hybridization frame proximate to the detection frame, and releasably mating the cover and the base.

For the purposes of this specification and appended claims, unless otherwise indicated, all numbers expressing quantities, percentages, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a substrate” includes two or more substrates. As used herein, the term “include” and its grammatical variants are intended to be non-limiting, such that recitation of items in a list or otherwise is not to the exclusion of other like items that can be substituted or added to the listed items.

It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

1-16. (cancelled).

17. In combination, a floating body provided with a hull, an earthmoving device, means for supporting the earthmoving device along the outside of the hull, and means for moving the earthmoving device in a longitudinal direction along the outside of the hull for the purpose of carrying out operations on the water bed.

18. The combination according to claim 17, wherein the earthmoving device comprises at least one sand extraction plant.

19. The combination according to claim 18, wherein the sand extraction plant comprises a pump that is drivable by a motor, a suction line that is in fluid connection with the pump for extracting a sand/water mixture, and a discharge pipe for discharging the extracted sand/water mixture.

20. The combination according to claim 17, wherein the earthmoving device is suspended, and is movable along the hull by at least one cable.

21. The combination according to claim 20, further comprising at least one hoisting device for paying out or hauling in the cable.

22. The combination according to claim 21, further comprising a second hoisting device, and a second cable interacting with said second hoisting device for paying out or hauling in the second cable.

23. The combination according to claim 21, wherein at least one hoisting device is fitted on the earthmoving device.

24. The combination according to claim 21, wherein at least one hoisting device is fitted on the floating body.

25. The combination according to claim 24, wherein at least one hoisting device is movable along the hull of the floating body.

26. The combination according to claim 21, wherein the earthmoving device comprises at least one arm having a free end on which a hoisting device is situated.

27. The combination according to claim 26, wherein the earthmoving device comprises a frame, and each arm is connected in a swiveling manner to the frame.

28. The combination according to claim 17, wherein the earthmoving device is suspended from, and is movable along, the outside of the hull in the longitudinal and transverse directions by a rigid guide.

29. The combination according to claim 28, wherein the rigid guide is a rail which extends around the outer periphery of the hull.

30. Earthmoving device, comprising:
at least one sand extraction plant provided with a pump that is drivable by a motor, a suction line in fluid communication with the pump for extracting a mixture of sand and water, and a discharge pipe for discharging the extracted mixture of sand and water;
at least one cable for suspending the earthmoving device along the outside of a hull of a floating vessel; and
means for moving the earthmoving device in the longitudinal and transverse directions along the outside of the hull.

31. The earthmoving device according to claim 30, further comprising at least one hoisting device for paying out or hauling in the cable.

32. The earthmoving device according to claim 31, further comprising at least one arm having a free end on which the hoisting device is situated.

33. The earthmoving device according to claim 32, further comprising a frame, and each arm is connected in a swiveling manner to the frame.

34. Method for moving a floating body in a body of water whose depth is less than the draft of the floating body, comprising the following steps:
providing an earthmoving device for carrying out operations on the bed of the body of water;
suspending the earthmoving device from the outside of the floating body;
moving the earthmoving device along the outside of the floating body; and
carrying out the operations on the bed of the body of water at several positions along the floating body, for restoring and/or retaining the floating power of the floating body.

35. The method according to claim 34, further comprising forming a deepened bed part next to and/or underneath the floating body.

36. The method according to claim 35, further comprising refloating or bringing ashore the floating body.
